(19) United States
(12) Patent Application Publication
(10) Pub. No.: US 2026/0208070 A1
(43) Pub. Date: Jul. 23, 2026

(54) METHOD FOR PRODUCING AN AQUEOUS SOLUTION OF 5-HYDROXYMETHYLFURFURAL COMPRISING A LIQUID-LIQUID EXTRACTION STEP INCORPORATING OR FOLLOWED BY FILTRATION

(71) Applicant: IFP Energies Nouvelles, Rueil-Malmaison (FR)

(72) Inventors: Meriem BOURAS, Rueil-Malmaison Cedex (FR); Olivier COTTE, Rueil-Malmaison Cedex (FR); Thi Bich Ngoc DANG, Rueil-Malmaison Cedex (FR); Maria FERNANDEZ ESPADA PASTOR, Rueil-Malmaison Cedex (FR); Kim LARMIER, Rueil-Malmaison Cedex (FR); Hélène LORCET, Rueil-Malmaison Cedex (FR); Alexandre PAGOT, Rueil-Malmaison Cedex (FR)

(73) Assignee: IFP Energies Nouvelles, Rueil-Malmaison (FR)

(21) Appl. No.: 19/138,853

(22) PCT Filed: Nov. 22, 2023
(86) PCT No.: PCT/EP2023/082709

§ 371 Date: Jun. 13, 2025

(30) Foreign Application Priority Data
Dec. 14, 2022 (FR) 2213367

(51) Int. Cl. B01D 11/04; C07D 307/46

(52) U.S. Cl. CPC B01D 11/0492; B01D 11/0488; C07D 307/46

(57) ABSTRACT

A process for producing an aqueous solution of 5-hydroxymethylfurfural (5-HMF), successively comprises a) bringing a feedstock (1) containing 5-HMF and a polar aprotic synthesis solvent into contact with an aqueous stream, b) a liquid-liquid extraction with an organic extraction solvent followed by backwashing c) with an aqueous solvent in order to obtain an organic raffinate rich in 5-HMF and in solvent. The organic raffinate, which is possibly concentrated, is then subjected to a hydrodistillation step e) in order to obtain an aqueous solution of 5-HMF. A step f) of liquid-solid separation of a solid particles fraction formed by precipitated humins and present within the aqueous raffinate (5) resulting from b) and/or of an intermediate liquid stream (19) resulting from b) is carried out. The aqueous raffinate resulting from liquid-liquid extraction b), freed of the-precipitated humins, is treated in a step g) of treating water-polar aprotic synthesis solvent mixtures.

### TECHNICAL FIELD

The invention relates to a process for producing an aqueous solution of 5-hydroxymethylfurfural (5-HMF).

### PRIOR ART

5-HMF is a compound of interest derived from biomass, which can be profitably exploited in many fields, especially in pharmaceuticals, agrochemistry or specialty chemicals. The production of 5-HMF by dehydration of sugars has been known for many years and has formed the subject of a large number of research studies.

Many dehydration conditions exist, and mention may in particular be made, by way of example, of the following methods:
5-HMF can be obtained in an aqueous medium, generally in the presence of an acid catalyst. This acid catalyst makes it possible to dehydrate the C6 sugar (in particular fructose) to give 5-HMF, but also catalyzes the rehydration of 5-HMF to give formic acid and levulinic acid, which is very harmful to the yield.
5-HMF can also be obtained in a nonaqueous polar protic medium, with solvents such as methanol, ethanol or acetic acid, and in the presence of an acid catalyst. Under these conditions, 5-HMF is obtained as a mixture with an ether or ester derivative of 5-HMF, depending on the reaction medium used. The formation of these byproducts is due to the reaction of 5-HMF with the reaction solvent in an acidic medium.
Patent application WO 2007/104514 describes the synthesis of 5-HMF by dehydration of sugar using methanol or ethanol as solvent in the presence of an acid catalyst. In this case, the presence of said catalyst also catalyzes the etherification reaction of 5-HMF with the alcohol to give a mixture of 5-HMF and its methyl or ethyl ether form, depending on the alcohol used as solvent.
5-HMF can also be produced in a polar aprotic medium, with or without an acid catalyst. Mention may more particularly be made of the use of dimethyl sulfoxide (DMSO) which, with or without an acid catalyst, makes it possible to produce 5-HMF with very good yields, and without the undesirable reactions listed above.

Furthermore, whatever the synthesis medium (water, methanol, DMSO, and the like), polymeric byproducts called humins are formed during the production of 5-HMF (van Dam, H. E.; Kieboom, A. P. G.; van Bekkum, H. (1986), The Conversion of Fructose and Glucose in Acidic Media: Formation of Hydroxymethylfurfural, In: Starch-Stärke, Vol. 38, No. 3, pages 95-101).

The synthesis of 5-HMF in a medium such as DMSO is particularly advantageous, as it makes it possible to obtain 5-HMF in its alcohol form (rather than the ether form) with very good yields. Nevertheless, the physicochemical properties of DMSO (or any other polar aprotic solvent) make it very difficult to separate from 5-HMF by the usual methods known to those skilled in the art.

One known method for isolating 5-HMF from DMSO is liquid-liquid extraction, followed by crystallization of the extract, as described in patent FR2669635. The applicant has already proposed an improvement to the process described in patent FR2669635, which was the subject of patent FR3071172. This improvement is based on the modification of the liquid-liquid extraction step, in particular by adding a step of backwashing with water, and by recycling the backwashing water upstream of the liquid-liquid extraction to mix it with the 5-HMF/DMSO feedstock, said mixture optionally being filtered before the liquid-liquid extraction. This improvement enables the purity of the 5-HMF to be increased without loss of yield of the product of interest, and enables the 5-HMF crystallization step to be carried out under more favorable conditions.

Nevertheless, despite the improvements afforded by patent FR3071172, the crystallization of 5-HMF remains a costly operation. The high production cost of 5-HMF limits its use, and the development of a process enabling a reduction in costs is needed.

In this context, the French patent application filed by the applicant under the number 2114335 discloses a process for recovering the 5-HMF not in crystallized form but in aqueous solution, in particular by implementing a step of concentrating an organic raffinate obtained in a step of backwashing an extract comprising 5-HMF resulting from a liquid-liquid extraction, and implementing a step of hydrodistilling the concentrated stream resulting from said concentration step, in order to recover 5-HMF in the form of an aqueous solution of 5-HMF. The process disclosed advantageously comprises a filtration step upstream of the liquid-liquid extraction to remove precipitated humins (solid particles) during the addition of water to the feedstock (step of mixing the feedstock with the backwashing water) before sending it to the liquid-liquid extraction. This is because the addition of water to the 5-HMF feedstock sent to the liquid-liquid extraction can cause the precipitation of humins present in the feedstock, which can generate operational problems during the liquid-liquid extraction, for example clogging the equipment.

The applicant has demonstrated another process which makes it possible to recover 5-HMF not in crystallized form but in aqueous solution, which, as for the process according to the French patent application filed under the number 2114335, opens up new possibilities for the exploitation of 5-HMF in various applications, or for subsequent transformations that could not be carried out either in DMSO or in the extraction solvent.

Moreover, the process according to the invention thus allows 5-HMF to be recovered in aqueous solution, while at the same time limiting the operating costs, water discharges and hence the environmental impact of said process. The process according to the invention also makes it possible to improve the removal of precipitated humins, in particular in order to protect the equipment used during the liquid-liquid extraction or during the downstream steps. This is because the problem of the precipitation of humins can also arise during the liquid-liquid extraction step and adversely affect the liquid-liquid extraction operation as well as downstream operations receiving streams that may comprise these precipitated humins, typically the treatment of water-DMSO mixtures resulting from the process.

### SUMMARY OF THE INVENTION

One subject of the present invention relates to a process for producing an aqueous solution of 5-HMF.

More particularly, the invention relates to a process for producing an aqueous solution of 5-hydroxymethylfurfural (5-HMF), said process comprising the following steps:
a step a) of bringing a feedstock comprising 5-HMF and a polar aprotic synthesis solvent into contact with an aqueous stream so as to obtain at least one aqueous mixture;
a step b) of liquid-liquid extraction of the aqueous mixture obtained on conclusion of step a) in the presence of an extraction solvent, so as to produce an aqueous raffinate comprising said polar aprotic synthesis solvent, an organic extract, a solid particles fraction, and optionally an intermediate liquid stream, said aqueous raffinate and/or said intermediate liquid stream comprising said solid particles fraction; then
a step c) of backwashing the organic extract with an aqueous solvent, so as to produce an intermediate aqueous back-extract and an organic raffinate comprising 5-HMF and an organic solvent;
an optional step d) of concentrating said organic raffinate resulting from step c) by removing at least a portion of the organic solvent, producing a concentrated organic raffinate comprising 5-HMF and residual organic solvent, and producing a first stream comprising organic solvent;
a hydrodistillation step e) implemented by distilling said organic raffinate resulting from step c) or said concentrated organic raffinate resulting from step d) in the presence of water, to produce an aqueous solution of 5-HMF and a second stream comprising organic solvent;
a step f) of liquid-solid separation of said solid fraction within said aqueous raffinate resulting from step b) and/or said intermediate liquid stream resulting from step b), producing a stream of solid particles and an aqueous raffinate depleted in solid particles and/or an intermediate liquid stream depleted in particles which is sent to step b);
a step g) of treating at least one water-polar aprotic synthesis solvent mixture produced in said process, said mixture consisting of said aqueous raffinate depleted in particles and resulting from step f) or of said aqueous raffinate not comprising the solid particles fraction and resulting from step b), to produce at least one aqueous effluent that can be recycled into said process.

According to one or more embodiments, step b) produces the intermediate liquid stream comprising the solid particles fraction, and said intermediate liquid stream is sent to step f) in order to separate the solid particles fraction from the intermediate liquid stream, forming the intermediate liquid stream depleted in particles which is sent to step b), and at least said water-polar aprotic synthesis solvent mixture consisting of said aqueous raffinate not comprising the solid particles fraction and resulting from step b) is sent to step g).

According to one or more embodiments, the intermediate liquid stream sent to step f) is an intermediate aqueous raffinate comprising the solid particles fraction and produced by separation between the extraction solvent and a water-polar aprotic synthesis solvent mixture in liquid-liquid extraction step b).

According to one or more embodiments, the intermediate liquid stream sent to step f) is a triphase mixture comprising a first liquid phase comprising 5-HMF and extraction solvent, a second liquid phase comprising water and polar aprotic synthesis solvent, and a solid phase comprising the solid particles fraction.

According to one or more embodiments, the aqueous raffinate produced in step b) comprises the solid particles fraction, and said aqueous raffinate is sent to step f) in order to separate the solid particles fraction from the aqueous raffinate, forming the aqueous raffinate depleted in solid particles which is sent to step g) as water-polar aprotic synthesis solvent mixture.

According to one or more embodiments, step f) is carried out at a temperature of between 0 and 60° C., and preferably comprises filtration, preferably carried out by a filter press.

According to one or more embodiments, step d) of concentrating the organic raffinate resulting from step c) comprises vaporization of the organic solvent at atmospheric pressure or under vacuum, preferably at a pressure of between 0.01 MPa and 0.1 MPa, and a liquid temperature maintained at less than or equal to 130° C., said concentrated organic raffinate comprising 5-HMF at a content of greater than or equal to 40% by weight and residual organic solvent at a content of less than or equal to 60% by weight.

According to one or more embodiments, step e) is carried out at atmospheric pressure or under vacuum, preferably at a pressure of between 0.001 MPa and 0.1 MPa, and preferably under vacuum at a pressure of between 0.005 MPa and 0.08 MPa.

According to one or more embodiments, step e) is implemented in a distillation column, preferably at a column bottom temperature of less than or equal to 140° C.

According to one or more embodiments, the extraction solvent is chosen from dichloromethane, diethyl ether, diisopropyl ether, methyl ethyl ketone, methyl isopropyl ketone, methyl isobutyl ketone, thiophene, anisole and toluene, and preferably is methyl isobutyl ketone.

According to one or more embodiments, the weight ratio (weight/weight) of aqueous solvent relative to the organic extract in backwashing step c) is between 0.04 and 5, preferably between 0.07 and 3, and preferably between 0.1 and 1.

According to one or more embodiments, the process comprises a step of dehydrating sugars to 5-HMF upstream of step a), preferably by bringing a sugar feedstock comprising one or more sugars into contact with said polar aprotic synthesis solvent and a dehydration acid catalyst, preferably at a temperature of between 30° C. and 200° C., preferably at a temperature of between 50° C. and 180° C., preferably between 70° C. and 150° C., preferably between 90° C. and 130° C., and at a pressure of between 0.001 MPa and 10 MPa, preferably between 0.001 MPa and 5 MPa, preferably between 0.01 MPa and 1 MPa.

According to one or more embodiments, the aqueous effluent produced in step g) is used in full or in part in step a) and/or in step c) and/or in step e).

According to one or more embodiments, in step a) the aqueous stream comprises all or a fraction of said intermediate aqueous back-extract resulting from step c).

According to one or more embodiments, the polar aprotic synthesis solvent is chosen from pyridine, butan-2-one, acetone, acetic anhydride, N,N,N′,N′-tetramethylurea, benzonitrile, acetonitrile, methyl ethyl ketone, propionitrile, hexamethylphosphoramide, nitrobenzene, nitromethane, N, N-dimethylformamide, N, N-dimethylacetamide, sulfolane, N-methylpyrrolidone, dimethyl sulfoxide, propylene carbonate and γ-valerolactone, taken alone or as a mixture, and is preferably dimethyl sulfoxide.

According to one or more embodiments, the concentrated organic raffinate obtained on conclusion of concentration step d) has a 5-HMF content of between 40% and 95% by weight and a residual organic solvent content of between 5% and 60% by weight, expressed relative to the weight of the concentrated organic raffinate.

According to one or more embodiments, an aqueous stream feeds the hydrodistillation step e).

According to one or more embodiments, step g) comprises treating one or more other water-polar aprotic synthesis solvent mixtures produced within the process.

Other subjects and advantages of the invention will become apparent on reading the description which follows of particular exemplary embodiments of the invention, which are given as non-limiting examples, the description being made with reference to the appended figures described below.

### LIST OF THE FIGURES

FIG. 1 illustrates one embodiment of the process according to the invention, wherein the liquid-solid separation step f) is carried out during the liquid-liquid extraction, on an intermediate liquid stream withdrawn in the liquid-liquid extraction step b).

FIG. 2 illustrates another embodiment of the process according to the invention, wherein the liquid-solid separation step f) is carried out downstream of the liquid-liquid extraction step b), on the aqueous raffinate obtained on conclusion of the liquid-liquid extraction step b).

FIG. 3 illustrates another embodiment of the process according to the invention, comprising, as for the embodiment illustrated in FIG. 1, a liquid-solid separation step f) carried out downstream of the liquid-liquid extraction step b), and comprising an additional liquid-solid separation step in mixing step a) and also various recycles of aqueous stream into the process.

In the figures, the same references denote identical or analogous elements.

### DESCRIPTION OF THE EMBODIMENTS

In the following detailed description, many specific details are disclosed in order to provide a deeper understanding of the process. However, it will be apparent to those skilled in the art that the process can be implemented without necessarily all of these specific details. In other cases, well-known features have not been described in detail in order to avoid unnecessarily complicating the description.

It is specified that, throughout this description, the expression “between . . . and . . . ” should be understood as including the limits mentioned, unless specified otherwise. In the present description, the term “to comprise” is synonymous with (means the same as) “to include” and “to contain”, and is inclusive or open-ended and does not exclude other elements which are not mentioned. It is understood that the term “to comprise” includes the exclusive and closed term “to consist of”.

For the purposes of the present invention, the various embodiments presented may be employed separately or in combination with each other, without any limit to their combinations where this is technically feasible.

For the purposes of the present invention, the various ranges of parameters for a given step, such as the pressure ranges and the temperature ranges, may be used alone or in combination. For example, for the purposes of the present invention, a preferred range of pressure values may be combined with a more preferred range of temperature values.

In the present description, an aprotic solvent is understood to mean a molecule which acts as solvent and all the hydrogen atoms of which are borne by carbon atoms.

In the present description, a polar solvent is understood to mean a molecule which acts as solvent, the dipole moment u of which, expressed in debye units, has a numerical value of greater than or equal to 2.00, measured at 25° C.

In the present description, a polar aprotic solvent is thus understood to mean a molecule which acts as solvent, all the hydrogen atoms of which are borne by carbon atoms and the dipole moment u of which, expressed in debye units, has a numerical value of greater than or equal to 2.00, measured at 25° C.

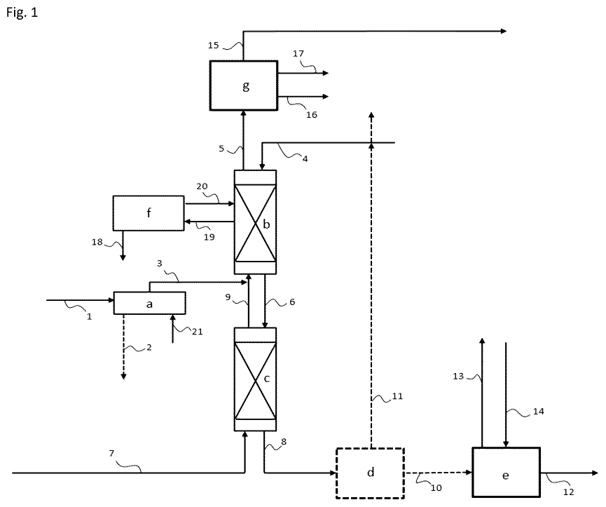

For better understanding of the invention, numerical references appearing in the figures are mentioned below to denote various elements of the process, without this constituting a limitation to the particular embodiments illustrated in FIGS. 1, 2 and 3.

### Optional Step of Dehydrating Sugars to 5-HMF

Advantageously, the feedstock 1 comprising 5-HMF and a polar aprotic synthesis solvent introduced in step a) according to the invention may be obtained during a step of dehydrating sugars to 5-HMF, very advantageously located upstream of step a) according to the invention, by bringing a sugar feedstock comprising one or more sugars into contact with a polar aprotic synthesis solvent and a dehydration acid catalyst so as to produce an effluent containing at least 5-HMF and a polar aprotic synthesis solvent, also referred to herein as synthesis effluent, and advantageously corresponding to the feedstock 1 of the process according to the invention introduced in mixing step a).

The process according to the invention may thus optionally comprise such a step of dehydrating sugars to 5-HMF, located upstream of step a).

The polar aprotic synthesis solvent is advantageously chosen from all the polar aprotic solvents of which the dipole moment expressed in debye (D) is greater than or equal to 2.00. Preferably, the polar aprotic solvents are chosen from pyridine (2.37), butan-2-one (5.22), acetone (2.86), acetic anhydride (2.82), N,N,N′,N′-tetramethylurea (3.48), benzonitrile (4.05), acetonitrile (3.45), methyl ethyl ketone (2.76), propionitrile (3.57), hexamethylphosphoramide (5.55), nitrobenzene (4.02), nitromethane (3.57), N,N-dimethylformamide (3.87), N,N-dimethylacetamide (3.72), sulfolane (4.80), N-methylpyrrolidone (4.09), denoted NMP, dimethyl sulfoxide (3.90), denoted DMSO, propylene carbonate (4.94) and γ-valerolactone (4.71) alone or as a mixture.

Preferably, the polar aprotic solvent is advantageously chosen from acetone, N,N-dimethylformamide, N,N-dimethylacetamide, sulfolane, NMP, DMSO, propylene carbonate and γ-valerolactone alone or as a mixture.

Preferably, the polar aprotic solvent is advantageously chosen from N,N-dimethylacetamide, NMP, DMSO, and γ-valerolactone, alone or as a mixture.

Very preferably, the polar aprotic solvent is DMSO.

The term “dehydration acid catalyst” is understood to mean any Brønsted acid catalyst chosen from organic or inorganic, homogeneous or heterogeneous Brønsted acids which are capable of inducing the dehydration of sugars to 5-HMF.

Preferably, the dehydration acid catalyst is a Brønsted acid having a pKa in the polar aprotic synthesis solvent, preferably in DMSO, of between 0 and 5.0, with preference of between 0.5 and 4.0 and preferably of between 1.0 and 3.0. Said pKa values are as defined in the article by F. G. Bordwell et al. (J. Am. Chem. Soc., 1991, 113, 8398-8401).

Preferably, the dehydration acid catalyst is chosen from HF, HCl, HBr, HI, H₂SO₃, H₂SO₄, H₃PO₂, H₃PO₄, HNO₂, HNO₃, H₂WO₄, H₄SiW₁₂O₄₀, H₃PW₁₂O₄₀, (NH₄)₆(W₁₂O₄₀).xH₂O, H₄SiMO₁₂O₄₀, H₃PMo₁₂O₄₀, (NH₄)₆Mo₇O₂₄.xH₂O, H₂MOO₄, HReO₄, H₂CrO₄, H₂SnO₃, H₄SiO₄, H₃BO₃, HClO₄, HBF₄, HSbF₅, HPF₆, H₂FO₃P, CISO₃H, FSO₃H, HN(SO₂F)₂, HIO₃, BF₃, AlCl₃, Al(OTf)₃, FeCl₃, ZnCl₂, SnCl₂, CrCl₃, CeCl₃, ErCl₃, formic acid, acetic acid, trifluoroacetic acid, lactic acid, levulinic acid, methanesulfinic acid, methanesulfonic acid, trifluoromethanesulfonic acid, bis(trifluoromethanesulfonyl)amine, benzoic acid, para-toluenesulfonic acid, 4-biphenylsulfonic acid, diphenyl phosphate and 1,1′-binaphthyl-2,2′-diyl hydrogen phosphate.

Preferably, the dehydration acid catalyst is chosen from HCl, H₂SO₄, H₃PO₂, H₃PO₄, HNO₃, AlCl₃, acetic acid, trifluoroacetic acid, methanesulfinic acid, methanesulfonic acid, and trifluoromethanesulfonic acid.

The term “sugar” denotes a sugar containing 6 carbon atoms (hexoses), but this does not exclude the presence in the feedstock of sugars containing 5 carbon atoms (pentoses), in the form of oligosaccharides and monosaccharides. In particular, the term “sugar” denotes glucose or fructose, alone or as a mixture, sucrose, but also oligosaccharides such as cellobiose, maltose, cellulose or even inulin.

The sugar feedstock employed may be sugar in solid form or else an aqueous solution of sugar known as syrup, preferably containing at least 30% by weight of sugar, more preferentially at least 50% by weight, and more preferentially still at least 70% by weight of sugar. By way of illustration, sucrose is generally produced in the form of a solid, while glucose or fructose, alone or as a mixture, are generally produced in the form of an aqueous solution (syrup), for example at 70% by weight of sugar.

The optional dehydration step is implemented at a temperature of between 30 and 200° C., with preference between 50 and 180° C., with preference between 70 and 150° C. and preferably between 90 and 130° C., for example a temperature of 120° C. Preferably, the optional dehydration step is performed at a pressure of between 0.001 MPa and 10 MPa, preferably between 0.001 MPa and 5 MPa, preferably between 0.01 MPa and 1 MPa. Depending on the pressure and temperature conditions, the reaction medium is above or below the bubble point of the mixture. The term “bubble point” denotes the pressure and temperature conditions at which the first gas bubbles are seen in a liquid. When the reaction medium is above the bubble point of the mixture, the vapor phase can be withdrawn from the reactor, optionally rectified, and condensed to form condensates which can be sent to a step g) for treating the water-polar aprotic synthesis solvent mixtures.

Preferably, the dehydration acid catalyst is introduced into the dehydration step in a molar ratio of the catalyst relative to the sugar feedstock, denoted Acid/Sugar, expressed as a molar percentage (mol %), of between 0.01 and 10 mol %, with preference between 0.05 and 8 mol %, with preference between 0.1 and 6 mol %, with preference between 0.2 and 5 mol %, preferably between 0.3 and 4 mol % and very preferably between 0.5 and 3 mol %.

The optional dehydration step may be performed according to various embodiments. Thus, the step may advantageously be implemented discontinuously or continuously (discontinuous mode being referred to as “batch mode”). The addition of the sugar feedstock may be progressive (fed-batch) in the case of a batch implementation, or staged in different CSTR reactors (Continuously Stirred Tank Reactor) in series in the case of a continuous implementation. The process may be performed in a closed reaction chamber or in a semi-open reactor.

Advantageously, the synthesis effluent obtained on conclusion of the optional dehydration step comprises 5-HMF and polar aprotic synthesis solvent, preferably DMSO. The polar aprotic synthesis solvent, typically DMSO, generally represents between 30% and 95% by weight of the synthesis effluent resulting from the dehydration step and treated in step a) of the process according to the invention, with preference between 40% and 90% by weight, with preference between 50% and 90% by weight, and preferably between 55% and 85% by weight.

The 5-HMF represents more than 1% by weight of the synthesis effluent resulting from the optional dehydration step and treated in step a) of the process according to the invention, with preference more than 10% by weight, with preference more than 15% by weight, and with preference less than 50% by weight, with preference less than 40% by weight, preferably less than 30% by weight.

Furthermore, said synthesis effluent resulting from the optional dehydration step may contain water even before being mixed in step a) with the aqueous stream 21. Said water may result from the dehydration step: for example, water is formed during the reaction of dehydration of sugar to 5-HMF (3 mol of water generated per mole of 5-HMF produced). This water may also have been introduced with the sugar, in the case where, for practical reasons, a sugar syrup, for example at about 70% by weight in water, is used. Advantageously, during the optional dehydration step, a water-polar aprotic synthesis solvent (e.g. DMSO) mixture may be recovered in the vapor phase. Said water-polar aprotic synthesis solvent (e.g. DMSO) mixture may advantageously be sent to step g). Thus, the synthesis effluent resulting from the optional dehydration step and introduced into step a) as feedstock 1 may contain water, in a proportion generally of between 0.1% and 30% by weight, preferably between 0.1% and 15% by weight, more preferably between 0.1% and 10% by weight.

It may be advantageous to carry out, during the optional dehydration step, a concomitant extraction of water from the reaction medium, in order to reduce the content of water in said reaction medium and thus improve the selectivity of the reaction. 5-HMF selectivity is understood to mean the ratio of the number of moles of 5-HMF produced to the number of converted moles of fructose which is contained in the sugar feedstock introduced into the process. In polar aprotic medium, the presence of water degrades the selectivity of sugar conversion in a manner that is all the more notable the higher the concentration of sugars in the DMSO. Such a continuous extraction of water during the synthesis of the 5-HMF is therefore advantageous in this respect, and also makes it possible to manage in a single step the extraction of the water that may be present in the sugar feedstock if it is in the form of a syrup. During such an extraction of water from the reaction medium, the reaction medium is above the bubble point of the mixture. The vapor phase may be withdrawn from the reactor, rectified and condensed to form water condensates which contain less than 10% by weight, preferably less than 5% by weight or even less than 1% by weight, of the polar aprotic synthesis solvent. The water extracted may result from the dehydration and/or have been introduced with the sugar feedstock, in the case where, for practical reasons, a feedstock in the form of syrup is used. Advantageously, at least 50% by weight, preferably at least 80% by weight, or even 90% by weight, of the water present in the reaction medium is extracted. The water extracted from the reaction medium may represent at least 50% by weight, preferably at least 80% by weight or even 90% by weight, of the water produced during the dehydration. The water may be extracted by various methods, such as evaporation, adsorption (for example in a molecular sieve), membrane separation or osmosis, and is advantageously extracted by distillation, which requires the polar aprotic synthesis solvent to be less volatile than water. Advantageously, the water is extracted under conditions which allow at least 90% by weight, preferably at least 95% by weight or even 99% by weight of the polar aprotic synthesis solvent employed in the dehydration step to be recovered in the synthesis effluent obtained on conclusion of the optional dehydration step. The water thus extracted may, like the aqueous effluent 15 resulting from step g) of treating the water-polar aprotic synthesis solvent mixtures, be recycled to one or more steps of the process requiring the supply of an aqueous stream, or be mixed with said recyclable aqueous effluent 15, or else be sent to step g) in order to be treated and remove the polar aprotic synthesis solvent that it may still contain, in order in particular to produce said recyclable aqueous effluent 15.

When the sugar feedstock is in the form of a syrup, it may be advantageous to reduce the content of water present upstream of the optional step of dehydrating the sugar feedstock, and optionally by extraction of the water resulting from the dehydration reaction during the dehydration step as already described above and not repeated here. Thus, it is possible to extract and replace the water of the sugar feedstock in the form of syrup with a polar aprotic synthesis solvent and obtain a mixture that is sent to the dehydration step. The water in the syrup may be extracted at least in part after mixing the syrup with the polar aprotic synthesis solvent. Said solvent makes it possible to keep the sugar in a diluted medium and to replace the dilution with water by the dilution with the synthesis solvent. The water may be extracted by various methods, such as evaporation, adsorption (for example in a molecular sieve), membrane separation, and is advantageously extracted by distillation, which requires the polar aprotic synthesis solvent to be less volatile than water. Advantageously, the extracted water represents at least 50% by weight, preferably at least 80% by weight, or even at least 90% by weight, of the water present in the syrup. The water extracted may represent between 90% and 99% by weight of the water of the syrup. Advantageously, the water extracted comprises less than 10% by weight, preferably less than 5% by weight or even less than 1% by weight, of synthesis solvent. The water thus extracted may, like the aqueous effluent 15 resulting from step g) of treating the water-polar aprotic synthesis solvent mixtures, be recycled to one or more steps of the process requiring the supply of an aqueous stream, or be mixed with said recyclable aqueous effluent 15, or else be sent to step g) in order to be treated and remove the polar aprotic synthesis solvent that it may still contain, in order in particular to produce said recyclable aqueous effluent 15.

The synthesis effluent resulting from the optional dehydration step and introduced into step a) as feedstock 1 may also contain impurities, in particular humins. The term “humins” refers to all of the undesirable polymeric compounds formed during the synthesis of the 5-HMF. In particular, the humins represent less than 30% by weight of the converted sugar feedstock, preferably less than 20% by weight.

An optional neutralization step may be carried out on the synthesis effluent resulting from the optional dehydration step before it is introduced into step a) as feedstock 1, said synthesis effluent containing the dehydration acid catalyst. This makes it possible to reduce the reactivity of the medium and thus avoid the mechanisms of degradation of the 5-HMF, or to reduce the corrosion of the materials of the equipment downstream of the optional dehydration step. Since the dehydration reaction may produce a few organic acids, the amount of neutralization agent may advantageously make it possible to neutralize all of the acids present in the synthesis effluent resulting from the dehydration step. Such a neutralization step is advantageously carried out at a minimum at the stoichiometric ratio of the amount of catalyst employed. Since the dehydration reaction may produce a few organic acids, the neutralization is generally carried out slightly superstoichiometrically with respect to the catalyst employed, preferentially between 1 and 2 times the stoichiometric ratio, preferentially between 1 and 1.5 the stoichiometric ratio. The neutralization agent may be a basic compound chosen from NaOH, KOH, NH₄OH, Na₂CO₃, K₂CO₃, NaHCO₃, KHCO₃, Mg(OH)₂, Ca(OH)₂, Ba(OH)₂.

### Mixing Step a)

The process according to the invention comprises a step a) of bringing the feedstock 1 comprising 5-HMF and a polar aprotic synthesis solvent, optionally resulting from the dehydration step, into contact (or mixing same) with an aqueous stream 21 so as to obtain at least one aqueous mixture 3.

The aqueous stream 21 may be composed of pure water, external to the process, or of water recycled from the process, for example the aqueous stream 21 may advantageously comprise all or some of the intermediate aqueous back-extract 9 resulting from step c) and/or of the water 15 produced in step g) of treating the water-polar aprotic synthesis solvent mixtures.

Preferably, the 5-HMF represents more than 1% by weight of the feedstock 1 introduced into step a) of the process according to the invention, with preference more than 10% by weight, with preference more than 15% by weight, and with preference less than 50% by weight, with preference less than 40% by weight, preferably less than 30% by weight.

Preferably, the polar aprotic synthesis solvent (e.g. DMSO) represents between 30% and 95% by weight of the feedstock 1 introduced into step a), with preference between 40% and 90% by weight, with preference between 50% and 90% by weight, and preferably between 55% and 85% by weight.

The feedstock 1 introduced into step a) may also contain water, in a proportion preferably of between 0.1% and 30% by weight, preferably between 0.1% and 15% by weight and more preferably between 0.1% and 10% by weight.

Feedstock 1 may optionally also contain humins. The humins represent, in particular, less than 30% by weight of feedstock 1, preferably less than 20% by weight.

The aqueous stream 21 therefore comprises, and may consist of, water. When the aqueous stream 21 comprises a fraction of recycled process water, said fraction may comprise at least 60% by weight of water, preferably at least 70% by weight, more preferentially at least 80% by weight, more preferentially still at least 95% by weight or even 98% by weight of water. The aqueous stream 21 may comprise all or a fraction of the intermediate aqueous back-extract 9 resulting from step c). Said intermediate aqueous back-extract 9 comprises water, polar aprotic synthesis solvent (e.g. DMSO) and optionally 5-HMF. Advantageously, said intermediate aqueous back-extract 9 contains more than 60% by weight of water, with preference more than 70% by weight of water and preferably more than 80% by weight of water.

Advantageously, the aqueous mixture 3 obtained on conclusion of step a) contains between 10% and 90% by weight of water, preferably between 20% and 80% by weight of water, and preferably between 40% and 75% by weight of water.

Preferably, step a) is carried out at a temperature of between 0 and 60° C., preferably of between 5 and 40° C., and is generally carried out at ambient temperature, i.e. at a temperature of between 10 and 40° C.

By increasing the water content of the feedstock 1 during step a), some of the humins present in the feedstock 1 can precipitate. The mixture resulting from the contacting of said feedstock 1 with aqueous stream 21 may therefore advantageously be subjected to a step of liquid-solid separation before being sent to the liquid-liquid extraction step b), so as to obtain a liquid separated from suspended solid particles and a solid residue comprising humins and which is preferably removed from the process in the form of a solid stream 2. Such an optional liquid-solid separation step thus makes it possible to remove the humins that have precipitated in step a) or upstream. At least a portion of the liquid obtained is then advantageously sent to the liquid-liquid extraction step b), said portion, or preferably all, of the liquid advantageously sent to step b) corresponding to the aqueous mixture 3. Such a liquid-solid separation step in step a) may advantageously be implemented when the amount of humins precipitated in the mixture formed by the feedstock 1 and the aqueous stream 21 in step a) is, for example, greater than 1% by weight. This optional liquid-solid separation step is preferably carried out at a temperature of between 0 and 60° C., preferably between 5 and 40° C., and generally at ambient temperature (i.e. between 10 and 40° C.). The optional liquid-solid separation step prior to step b) is a simple solid-liquid separation, and may be carried out by any method known to those skilled in the art, such as by using a filter press, a belt filter, a clarifier, a settler, a centrifuge, for example a plate centrifuge, said techniques being used alone or in combination, in any order. Preferably, the liquid-solid separation step is a filtration, preferably carried out with a filter press.

### Liquid-liquid Extraction Step b)

The process according to the invention comprises a step b) of liquid-liquid extraction of the aqueous mixture 3 obtained on conclusion of step a) in the presence of an extraction solvent 4, so as to produce an aqueous raffinate 5 and an organic extract 6. The liquid-liquid extraction carried out in step b) advantageously corresponds to washing the aqueous mixture with an organic extraction solvent. Preferably, the liquid-liquid extraction carried out in step b) is a countercurrent extraction of the aqueous mixture 3 obtained in step a) with an extraction solvent 4. This technique is well known to those skilled in the art. It may be carried out, for example, in a mixer-decanter array, in a column filled with random or structured packing, in a pulsed column, or even in a stirred column.

The liquid-liquid extraction may comprise the implementation of at least two theoretical separation stages. This is the case, for example, when the liquid-solid separation step f) is carried out on intermediate liquid stream 19 resulting from the liquid-liquid extraction step b), which returns to step b) once freed of the solid particles, as detailed further below in step f). A first liquid-liquid separation stage may then be carried out, producing said intermediate liquid stream 19 sent to the liquid-solid separation step f) to form an intermediate liquid stream depleted in particles 20 which is sent to the second liquid-liquid extraction stage of step b).

The liquid-liquid extraction step b) is advantageously carried out at a temperature of between 0 and 60° C., preferably between 5° C. and 40° C., and generally at ambient temperature (i.e. between 10 and 40° C.).

The weight proportion (weight/weight) of extraction solvent 4 relative to the aqueous mixture 3 is preferably between 0.2 and 5, preferably between 1 and 3, and preferably between 1.5 and 2.5.

The extraction solvent 4 introduced into step b) is chosen from water-immiscible organic solvents, so as to form two liquid phases in step b) and in backwashing step c). This property is highly dependent on the relative proportion of the flow rates of feedstock, of back-extraction water and of extraction solvent used in the process.

In a nonlimiting manner, the extraction solvent is preferably chosen from chlorinated organic solvents, ethers, esters, ketones and aromatic compounds. Preferably, the extraction solvent is a chlorinated solvent having between 1 and 10 carbon atoms, denoted hereafter as C1-C10, an ether having between 2 and 10 carbon atoms (C2-C10), an ester having between 4 and 10 carbon atoms (C4-C10), a ketone having between 3 and 10 carbon atoms (C3-C10), an aldehyde having between 1 and 10 carbon atoms (C1-C10), or a C4-C10 aromatic compound. Preferably, the extraction solvent is chosen from dichloromethane, diethyl ether, diisopropyl ether, methyl ethyl ketone, methyl isopropyl ketone, methyl isobutyl ketone, thiophene, anisole and toluene. Very preferably, the extraction solvent is methyl isobutyl ketone.

Advantageously, the extraction solvent is chosen so as:
to have a very great difference in volatility with 5-HMF, in particular so as to facilitate its removal in optional step d) and to limit the degradation of 5-HMF, i.e. so as to have in step d) a vaporization rate which avoids degradation of 5-HMF and minimizes the amount of residual solvent to be removed in step e), while at the same time ensuring the absence of liquid phase separation when the concentrated organic raffinate 10 is brought into contact with water in step e), and
to form in step e) a heterogeneous azeotrope with water, which is preferably solvent-rich, i.e. with more than 50% by weight of solvent, with preference with more than 60% by weight of solvent and preferably with more than 70% by weight of solvent. Advantageously, said azeotrope of the water/extraction solvent mixture has a boiling temperature significantly lower than that of water, with preference at least 5° C. lower than the boiling temperature of water, with preference at least 8° C. lower than the boiling temperature of water and preferably at least 10° C. lower than the boiling temperature of water.

Advantageously, the organic solvent streams produced in the subsequent steps may be recycled to the extraction step b), as extraction solvent. These organic solvent streams may contain impurities which may have been generated during the implementation of the process. Advantageously, the organic solvent streams produced in the subsequent steps may be distilled, for example periodically, to avoid accumulation of said impurities.

Step b) thus makes it possible to obtain, firstly, an aqueous stream depleted in 5-HMF, called aqueous raffinate 5, which contains a large part of the polar aprotic synthesis solvent (e.g. DMSO) initially contained in the feedstock 1, and secondly an organic stream enriched in 5-HMF, called organic extract 6, which contains a large part of the 5-HMF, initially contained in the feedstock 1, and the extraction solvent 4. This organic extract 6 may also contain polar aprotic synthesis solvent (e.g. DMSO). Preferably, said organic extract preferably contains 5-HMF and polar aprotic synthesis solvent (e.g. DMSO) in a 5-HMF/polar aprotic synthesis solvent (e.g. DMSO) weight ratio of between 50/50 and 99/01, with preference of between 50/50 and 95/05, with preference of between 55/45 and 90/10, more preferentially of between 60/40 and 85/15 and preferably of between 65/35 and 80/20.

Advantageously, the organic extract 6 is sent directly to the backwashing step c).

A solid particles fraction, formed by precipitated humins, is also present in step b). These may be humins precipitated in the upstream mixing step a), still present in the aqueous mixture 3 sent to step b), or humins precipitated in step b) or in the backwashing step c).

Said solid particles fraction may be in suspension in the intermediate liquid stream 19 optionally formed in step b) and/or in the aqueous stream 15. When said aqueous stream 15 comprises the solid particles fraction, the aqueous stream 15 is sent to liquid-solid separation step f) described further below.

### Backwashing Step c)

The process according to the invention comprises a step c) of backwashing the organic extract 6 with an aqueous solvent 7, so as to produce an intermediate aqueous back-extract 9 and an organic raffinate 8 comprising 5-HMF and an organic solvent. The intermediate aqueous back-extract 9 is advantageously sent partly or totally into step a). The organic solvent is in particular composed at least partly of extraction solvent and may optionally comprise polar aprotic synthesis solvent (e.g. DMSO), preferably in small amounts.

The introduction of an aqueous solvent 7 in step c) is carried out so as to implement backwashing, according to the general knowledge of those skilled in the art.

The introduction of the aqueous solvent 7 is carried out such that the amount of aqueous solvent is as low as possible in order to reduce costs, but sufficient to ensure a low weight content of polar aprotic synthesis solvent (e.g. DMSO) in the organic raffinate 8, and with preference less than or equal to 20.0% by weight relative to the weight of the 5-HMF, preferentially less than or equal to 15.0% by weight relative to the weight of the 5-HMF, preferably between 0.01% and 15.0% by weight relative to the weight of the 5-HMF, very preferably between 0.01% and 10.0% by weight relative to the weight of the 5-HMF.

Advantageously, the aqueous backwashing solvent 7 introduced in step c) comprises at least 95% by weight of water, preferably at least 98% by weight of water (100% being the maximum).

The aqueous solvent may optionally comprise polar aprotic synthesis solvent (e.g. DMSO). The backwashing efficacy is all the higher the lower the amount of polar aprotic synthesis solvent (e.g. DMSO) present in the aqueous backwashing solvent. The aqueous solvent may comprise at most 1.0% by weight and preferably at most 0.1% by weight of polar aprotic synthesis solvent (e.g. DMSO). Advantageously, the aqueous backwashing solvent 7 results from step g) of treating water-polar aprotic synthesis solvent mixtures produced within the process, and thus comprises at least a fraction of the recyclable effluent 15. In a preferred embodiment of the invention, the aqueous raffinate 5 composed of water and polar aprotic synthesis solvent (e.g. DMSO), produced in step b), or the aqueous raffinate depleted in particles 5′ and produced in step f), is treated in step g), which advantageously comprises a distillation. The water-rich distillate thus obtained on conclusion of this step g), also referred to as aqueous effluent 15 recyclable into the process, is advantageously used to form the aqueous backwashing solvent 7 in step c), optionally as a mixture with a makeup of water 22, or is used in mixing step a) to form the aqueous stream 21, optionally with at least one intermediate aqueous back-extract 7 fraction and/or a makeup of water. Said recyclable effluent 15, e.g. water-rich distillate, may also contain a residual amount of polar aprotic synthesis solvent (e.g. DMSO), with preference of less than or equal to 1% by weight and preferably of less than or equal to 0.1% by weight. The residual amount of polar aprotic synthesis solvent (e.g. DMSO) in the aqueous effluent 15 (the distillate) is proportionately lower the more efficiently the distillation of step g) is carried out, in particular with a number of distillation stages of greater than 5, and advantageously suitable boil-up rates and reflux ratios.

The backwashing step c) is advantageously a liquid-liquid extraction of an organic stream, in particular the organic extract 6 obtained in step b) in countercurrent to the aqueous solvent 7. This technique is well known to those skilled in the art. The extraction may be carried out, for example, in a mixer-decanter array, in a column filled with random or structured packing, in a pulsed column, or even in a stirred column.

Step c) is preferably carried out at a temperature of between 0 and 60° C., preferably between 5 and 40° C., and generally at ambient temperature (i.e. between 10 and 40° C.).

The weight ratio (weight/weight) of aqueous solvent 7 relative to the organic extract 6 is preferably between 0.04 and 5, preferably between 0.07 and 3, and preferably between 0.1 and 1.

Step c) makes it possible to obtain an aqueous stream advantageously enriched in polar aprotic synthesis solvent (e.g. DMSO), referred to as intermediate aqueous back-extract 9, preferably containing at least 60% by weight of water, preferably at least 80% by weight of water, and an organic raffinate 8, advantageously depleted in polar aprotic synthesis solvent (e.g. DMSO). Said intermediate aqueous back-extract 9 is advantageously sent, partly or preferably totally, to step a). The organic raffinate 8 obtained has a weight content of polar aprotic synthesis solvent preferably of less than or equal to 20.0% by weight relative to the weight of the 5-HMF, preferably of less than or equal to 15.0% by weight, more preferentially of less than or equal to 5.0% by weight, even more preferentially of less than or equal to 4.0% by weight, and more preferably of less than or equal to 3.0% by weight, relative to the weight of the 5-HMF.

According to the invention, the organic raffinate 8 produced in step c) is sent to the optional concentration step d) or directly to the hydrodistillation step e).

Humins may still be present in the organic extract 6 sent to the backwashing step c). In the event that they precipitate in this step, then forming undesirable solid particles, these could be removed in the liquid-solid separation step f) by sending to step b), in full or in part, the intermediate aqueous back-extract 9, which would include the precipitated humins, which could be sent to liquid-solid separation step f) with the intermediate liquid stream 19 and/or the aqueous raffinate 5. In the event that the intermediate aqueous back-extract 9 is partly or totally sent to step a) to enter the composition or constitute the aqueous stream 21, the precipitated humins could also be separated during the optional liquid-solid separation of step a), or else in step f) as already described above and detailed further below.

### Optional Concentration Step d)

The process according to the invention preferentially comprises a step d) of concentrating the organic raffinate 8 resulting from step c), by removing a portion of the organic solvent, producing a concentrated organic raffinate 10 comprising 5-HMF and residual organic solvent, and a first stream 11 comprising, and preferably consisting of, organic solvent, said organic solvent advantageously being composed fully or partially of the extraction solvent and optionally of polar aprotic synthesis solvent (e.g. DMSO).

Preferably, the first stream 11 comprising organic solvent is recycled, in full or in part, to the extraction step b), for example coming to form at least a portion of the organic solvent stream 4.

Preferably, in step d), the removal of a portion of the organic solvent is performed by vaporization, for example in an atmospheric-pressure or vacuum distillation column, in an evaporator, or via any method known to those skilled in the art.

According to this preferred embodiment, the vaporization of the organic solvent is advantageously carried out at atmospheric pressure or under vacuum, preferably at a pressure of between 0.01 MPa and 0.1 MPa, preferentially under vacuum at a pressure of between 0.01 MPa and 0.09 MPa, so as to limit the temperature of the liquid and thus the degradation of the 5-HMF. Preferably, the temperature of the liquid is maintained at less than or equal to 130° C., preferably maintained at less than or equal to 100° C., preferably maintained at less than or equal to 70° C. The pressure level, especially vacuum level, to be applied to reach these temperatures is of course dependent on the organic solvent and more particularly on the extraction solvent used and the organic solvent vaporization rate.

In a preferred embodiment, the vaporization of the solvent is carried out by multi-effect evaporation or mechanical vapor recompression, or any other method known to those skilled in the art, so as to reduce the operating costs associated with the evaporation of the solvent while limiting the risks of degradation of the product of interest, i.e. the 5-HMF. For example, in the case of a triple-effect evaporator, the temperature of the liquid is kept below 130° C. in the first effect, below 100° C. in the second effect, and below 70° C. in the third effect. Thus, the temperature of the liquid phase is progressively reduced as the 5-HMF is concentrated in the organic solvent, limiting any risk of degradation.

Optional step d) is implemented with a vaporization mass rate (or evaporation rate), corresponding to the mass of organic solvent vaporized relative to the mass of organic raffinate 8 resulting from step c) (more particularly the amount by mass of stream 11 relative to the amount by mass of organic raffinate 8), of at least 50%, with preference of at least 60%, with preference of at least 70%, with preference of at least 75%, with preference of at least 80%, with preference of at least 85%, preferably of at least 90%, and with preference of at most 99%. Advantageously, the vaporization rate is defined as according to the extraction solvent so as not to degrade the 5-HMF, but also so as to minimize the amount of residual solvent to be removed in step e), while at the same time ensuring the absence of liquid phase separation (i.e. while ensuring that the liquid phase remains single-phase) when the concentrated organic raffinate 10 is brought into contact with water in step e).

By virtue of the combination of all of the operating conditions of the preceding steps a), b) and c), and of the optional step d), the concentrated organic raffinate 10 obtained on conclusion of step d) very advantageously has a 5-HMF content of at least 40% by weight relative to the weight of the concentrated organic raffinate, with preference at least 50% by weight, with preference at least 60% by weight, and with preference at most 95% by weight, with preference at most 90% by weight and preferably at most 85% by weight, relative to the weight of the concentrated organic raffinate 10. In other words, the concentrated organic raffinate 10 preferably has a residual organic solvent content of at least 5% by weight relative to the weight of the concentrated organic raffinate, preferably at least 10% by weight, and preferably at most 60% by weight, preferably at most 50% by weight, preferably at most 40% by weight, relative to the weight of the concentrated organic raffinate 10.

Advantageously, the organic solvent vaporized during the optional step d) forms a first stream 11 comprising, preferably consisting of, organic solvent and is preferably recycled to the extraction step b).

Advantageously, the concentrated organic raffinate 10 is sent to the hydrodistillation step e).

### Hydrodistillation Step e)

The process according to the invention comprises a hydrodistillation step e) implemented by distilling the concentrated organic raffinate 10 resulting from optional step d) or the organic raffinate 8 resulting from step c) in the presence of water, so as to produce an aqueous solution 12 of 5-HMF and a second stream 13 comprising, and preferably consisting of, organic solvent.

The hydrodistillation step e) advantageously enables the residual organic solvent not removed during the optional step d) to be at least partly removed. The residual organic solvent removed during step e), i.e. the second stream 13 comprising organic solvent, may advantageously be recycled to the extraction step b), alone or as a mixture with the first stream 11 resulting from the optional step d).

Advantageously, an aqueous liquid 14 feeds the hydrodistillation step e). The aqueous liquid 14 introduced in step e) preferably contains more than 95% by weight of water, preferably more than 98% by weight of water.

In one particular embodiment of the invention, the aqueous liquid 14 is pure water, possibly external to the process, which allows the content of residual polar aprotic synthesis solvent (e.g. DMSO) in the aqueous 5-HMF solution 12 produced in step e) to be minimized even further.

In another particular embodiment of the invention, water isolated within the process is used to feed step e), making it possible to limit the process operating costs and its environmental impact. Typically, if the process integrates the preparation of the feedstock 1 and the sugar feedstock for the dehydration step is a sugar syrup at 70% by weight in water, about 1 ton of water is present on conclusion of the dehydration step (the water in the sugar feedstock and the water produced during the dehydration reaction) per ton of 5-HMF produced. This water, which is advantageously recovered, needs to be treated before being discharged into the environment. The process according to the invention may then advantageously use said water resulting from the sugar feedstock and/or the dehydration step to produce on conclusion of step e) an aqueous 5-HMF solution concentrated preferably to 30% by weight or more, preferentially to 40% by weight or more, and thus to reduce the reprocessing costs of the process and its environmental impact.

Advantageously, the aqueous liquid 14 introduced into step e) may correspond to at least a fraction, optionally all, of the aqueous effluent 15 (distillate) produced in step g). Said distillate may optionally contain a residual amount of polar aprotic synthesis solvent (e.g. DMSO).

Advantageously, during step e), the extraction solvent employed in the process forms a heterogeneous azeotrope with water, said azeotrope preferably being rich in extraction solvent, with preference comprising more than 50% by weight of extraction solvent, with preference more than 60% by weight of extraction solvent and preferably more than 70% by weight of extraction solvent. Advantageously, said water/extraction solvent azeotrope has a boiling temperature significantly lower than that of water, with preference at least 5° C. lower than the boiling temperature of water, with preference at least 8° C. lower than the boiling temperature of water and preferably at least 10° C. lower than the boiling temperature of water.

Thus, after bringing the concentrated organic raffinate 10 or the organic raffinate 8 into contact with the aqueous liquid 14, the residual organic solvent can be easily removed without degradation of the 5-HMF.

The hydrodistillation step e) may be carried out at atmospheric pressure or under vacuum and in particular at a pressure of between 0.001 MPa and 0.1 MPa, and preferably under vacuum at a pressure of between 0.005 MPa and 0.08 MPa. Advantageously, the hydrodistillation step is implemented under vacuum, in particular at a pressure of between 0.001 MPa and 0.1 MPa, preferably between 0.005 MPa and 0.08 MPa, so as to facilitate removal of the residual organic solvent without degradation of the 5-HMF.

Advantageously, the hydrodistillation step e) is implemented in a distillation column, preferably at a column bottom temperature of less than or equal to 140° C., with preference of less than or equal to 130° C., with preference of less than or equal to 120° C., with preference of less than or equal to 110° C. and preferably of less than or equal to 100° C., so as to facilitate removal of the residual organic solvent without degradation of the 5-HMF.

In one particular embodiment, the concentrated organic raffinate 10 or otherwise the organic raffinate 8 and the aqueous liquid 14 are mixed prior to introduction into a distillation column and the mixture is introduced at an intermediate point of the distillation column.

In another particular embodiment, the concentrated organic raffinate 10 or the organic raffinate 8 is introduced into the upper part of the distillation column, preferably into the upper half of the distillation column, while the aqueous liquid is also introduced into the distillation column. The mixing with the aqueous liquid is then carried out within the distillation column.

Given the formation of a heterogeneous azeotrope between the water and the extraction solvent, condensation of the distillation column overhead vapors generates two liquid phases: a water-rich phase which may advantageously be returned to the column as reflux, and an organic solvent-rich phase 13 which may advantageously be recycled into the extraction step b).

According to the invention, the aqueous 5-HMF solution 12 obtained on conclusion of step e) has an amount of 5-HMF of at least 30% by weight, with preference at least 40% by weight, and with preference less than 90% by weight, with preference less than 85% by weight and preferably less than 80% by weight, the percentages being given by weight of 5-HMF relative to the weight of aqueous 5-HMF solution obtained on conclusion of step e).

The process according to the invention thus enables the production of an aqueous 5-HMF solution very advantageously having a weight content of polar aprotic synthesis solvent (e.g. DMSO) of less than or equal to 10% by weight relative to the weight of 5-HMF, with preference of less than or equal to 5% by weight relative to the weight of 5-HMF and preferably of less than or equal to 3% by weight relative to the weight of 5-HMF.

### Liquid-solid Separation Step f)

According to the invention, the process comprises a liquid-solid separation step f) making it possible to reduce the content of solid particles formed by precipitated humins on at least one of the intermediate liquid stream 19 or of the (final) aqueous raffinate 5 which result from the liquid-liquid extraction step b).

The liquid-liquid extraction step b) may induce precipitation of solid, i.e. precipitated humins, independently of that which can occur in step a) or upstream of step a), and a solid particles fraction generated during the liquid-liquid extraction or not removed by a possible liquid-solid separation in step a) may cause the problems already described. In order to preserve the equipment downstream of the liquid-liquid extraction step, and in particular the equipment employed in step g) of treating the water-polar aprotic synthesis solvent mixtures and possibly at the end of the liquid-liquid extraction, a separation of the precipitated solid proves to be necessary.

Said separation is carried out on the intermediate liquid stream 19 if this is produced in step b), or on the (final) aqueous raffinate 5 produced in the liquid-liquid extraction step b), or on both.

This liquid-solid separation step f) is preferably carried out at a temperature of between 0 and 60° C., preferably between 5° C. and 40° C., and generally at ambient temperature (i.e. between 10° C. and 40° C.).

The liquid-solid separation step is preferably a simple liquid-solid separation, and may be carried out by any method known to those skilled in the art, for example employing a filter press, a belt filter, a clarifier, a settler, a centrifuge, for example a plate centrifuge, it being possible for said separation techniques to be used alone or in combination, in any order. Preferably, the liquid-solid separation step is a filtration, preferably carried out with a filter press.

The liquid-solid separation in step b) may comprise the addition of an additive facilitating liquid-solid separation, for example the addition of diatomaceous earths in the case of filtration in order, for example, to accelerate the filtration or to improve the filtration in the case where the solid particles are too sticky.

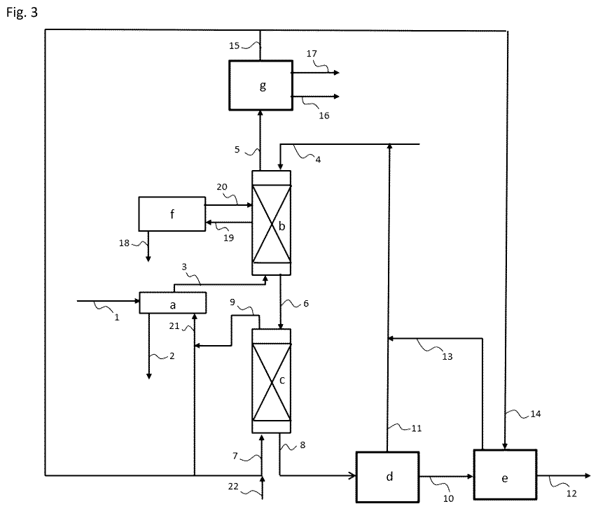

According to one or more embodiments, such as represented for example in FIG. 1 or in FIG. 3, step b) produces an intermediate liquid stream 19 comprising said solid particles fraction, and said intermediate liquid stream 19 is sent to step f) in order to separate said solid particles fraction from said 19, and to form an intermediate liquid stream depleted in particles 20 which is sent to step b), typically to a second liquid-liquid extraction stage. The final aqueous raffinate 5 resulting from step b) and sent to step g) does not comprise the solid particles fraction resulting from step b); the precipitated humins have in fact been removed by carrying out the separation on the intermediate liquid stream 19.

According to one or more embodiments, said intermediate liquid stream 19 sent to step f) is an intermediate aqueous raffinate comprising the solid particles fraction, which is produced by separation between the extraction solvent and a water-polar aprotic synthesis solvent mixture in the liquid-liquid extraction step b).

Alternatively, the intermediate liquid stream 19 sent to step f) is a triphase mixture comprising a first liquid phase comprising 5-HMF and extraction solvent (which may be denoted as intermediate extract), a second liquid phase comprising water and polar aprotic synthesis solvent (which may be denoted as intermediate raffinate), and a solid phase comprising the solid particles fraction.

A three-phase centrifugal separator may then advantageously be used to separate, in a single piece of equipment, the 3 phases: intermediate raffinate, intermediate extract and solid phase comprising the precipitated humins (stream of solid particles 18). Such equipment used for step f) may be that used in a stage of the liquid-liquid extraction b). Intermediate raffinate and intermediate extract come to form the aqueous raffinate 5 and the organic extract 6, respectively.

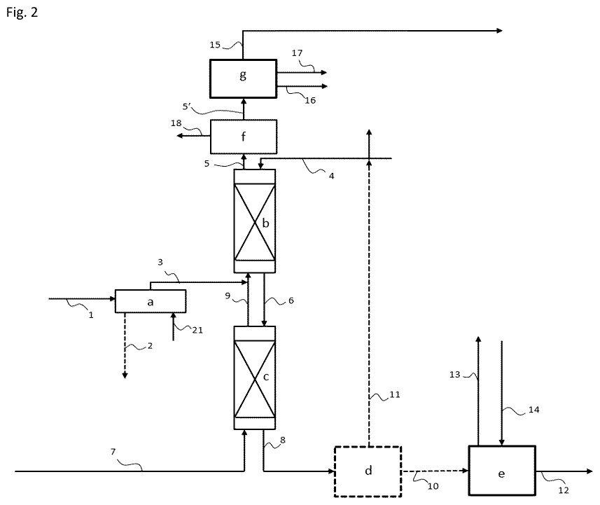

According to one or more embodiments, such as for example represented in FIG. 2, the aqueous raffinate 5 produced in step b) comprises said solid particles fraction, and said aqueous raffinate 5 is sent to step f) in order to separate the solid particles fraction from the aqueous raffinate 5, and form an aqueous raffinate depleted in solid particles 5′which is sent to step g) as water-polar aprotic synthesis solvent mixture.

According to one or more embodiments, step f) is carried out both on the intermediate liquid stream 19 and on the final aqueous raffinate 5 that are produced in step b), separately, if these two effluents each contain solid particles formed by the precipitated humins. In this case, an aqueous raffinate depleted in solid particles is produced and sent to step g) as a water-polar aprotic synthesis solvent mixture.

Intermediate liquid raffinate/intermediate aqueous raffinate/aqueous raffinate depleted in solid particles (precipitated humins) is understood to mean a raffinate comprising less than 10% by weight of solid particles formed by the precipitated humins, preferably less than 5% by weight, and more preferentially less than 1% by weight.

### Step g) of Treating Water-polar Aprotic Synthesis Solvent Mixtures

The process according to the invention comprises a step g) of treating water-polar aprotic synthesis solvent (e.g. DMSO) mixtures generated by the steps of the process according to the invention, to produce an aqueous effluent (also referred to as distillate), which may be used in full or in part in the backwashing step c) and/or in step a) and/or in step e). This step may also produce a stream 16 rich in polar aprotic synthesis solvent (e.g. DMSO) and a stream of impurities 17. At least one water-polar aprotic synthesis solvent mixture produced in the process is therefore treated in step g). Said mixture may be the aqueous raffinate depleted in particles 5′resulting from step f) or the aqueous raffinate 5 not comprising the solid particles fraction resulting from step b).

This step specifically makes it possible to separate, within said at least one mixture (aqueous raffinate depleted in particles 5′resulting from step f) or aqueous raffinate 5 not comprising the solid particles fraction resulting from step b)), the water, the polar aprotic synthesis solvent and reaction products extracted in the raffinate such as unconverted sugars, sugar oligomers, residual 5-HMF.

One or more other water-polar aprotic synthesis solvent mixtures produced within the process may be sent to step g).

If the process integrates a step of synthesizing the feedstock 1, i.e. a step of dehydrating a sugar feedstock as described above, and (i) this step comprises a concomitant extraction of water from the reaction medium or (ii) this reaction step of dehydrating a sugar feedstock is preceded by a step of separating the water contained in the sugar feedstock, in the case where the latter is initially in the form of a syrup, comprising extracting and substituting the water of the syrup with polar aprotic synthesis solvent, then the stream rich in synthesis solvent 16 may be purged of its water at the same time as the extraction of the water in these two scenarios (i) and (ii). The residual amount of polar aprotic synthesis solvent (e.g. DMSO) in the aqueous effluent produced on conclusion of step g) is proportionately lower the more the distillation is carried out efficiently according to the knowledge of those skilled in the art.

The water-polar aprotic synthesis solvent (e.g. DMSO) mixtures generated by the process denote in particular the aqueous raffinate 5 produced in step b) and depleted in solid particles in step f), and optionally the water-polar aprotic synthesis solvent (e.g. DMSO) mixture resulting from the optional step of dehydrating sugars to 5-HMF when the process integrates such a step.

Step g) of treating the water-polar aprotic synthesis solvent (e.g. DMSO) mixtures preferably employs a section for evaporating a water-polar aprotic synthesis solvent (e.g. DMSO) mixture, to remove any impurities in the form of the stream 17, in particular heavy impurities such as humins or unconverted sugars, followed by a distillation section.

The evaporation section is operated at a temperature preferably of between 80 and 120° C., preferentially between 100 and 110° C., and preferably at a pressure between 0.002 MPa and 0.020 MPa, preferentially between 0.005 MPa and 0.010 MPa. Preferably, the evaporation section employs an evaporator of wiped film type (Thin Film Evaporator, TFE).

The distillation section for its part advantageously employs a distillation column or several separate items of equipment. Preferably, the distillation section of step g) is advantageously implemented in a distillation column, at a column top temperature preferably of between 25 and 60° C., preferentially between 45 and 55° C., for example about 50° C., preferably at a column bottom temperature of between 80 and 140° C., preferentially between 100 and 130° C., for example about 120° C., with preference at a pressure of between 0.001 MPa and 0.05 MPa, preferentially between 0.005 MPa and 0.02 MPa and preferably between 0.008 MPa and 0.012 MPa, and with preference with a reflux ratio of between 0.01 and 0.50, preferably between 0.05 and 0.10.

Thus, the aqueous raffinate 5 produced in step b) and comprising water and polar aprotic synthesis solvent (e.g. DMSO) and optionally the water-polar aprotic synthesis solvent (e.g. DMSO) mixture recovered in the optional dehydration step are evaporated, then the gas phase is recovered and distilled, preferably under vacuum, so as to produce a residue 16 rich in polar aprotic synthesis solvent (e.g. DMSO) on the one hand, a water-rich distillate 15 (corresponding to the aqueous effluent) on the other hand, and, lastly, a stream 17 containing heavy fractions such as humins not removed by filtration and unconverted sugars of humins. The term “rich” here is understood to mean at least 95% by weight, preferably at least 98% by weight. Some or all of the water-rich distillate, or aqueous effluent, may advantageously be recycled to step c) as aqueous solvent for carrying out the backwashing step and/or to the hydrodistillation step e) as aqueous stream. Said water-rich distillate may also be totally or partly recycled as water introduced into step a).

The residue rich in polar aprotic synthesis solvent (e.g. DMSO) may advantageously be introduced into the optional dehydration step, either directly or after distillation enabling any heavy products that might accumulate to be discharged.

The example and figures described in detail below illustrate the invention without limiting its scope.

FIG. 1 illustrates one particular embodiment of the process according to the invention. The feedstock 1 containing 5-HMF, polar aprotic synthesis solvent (e.g. DMSO) and humins is sent to step a) and is brought into contact with an aqueous stream 21. The aqueous mixture 3 obtained on conclusion of step a) is sent to the extraction step b) and brought into the presence of an extraction solvent 4 in order to extract the 5-HMF from the aqueous mixture using the extraction solvent and obtain an aqueous raffinate 5 and an organic extract 6. The organic extract 6 is brought into the presence of an aqueous solvent 7 in the backwashing step c). The organic raffinate 8 obtained on conclusion of step c) may be concentrated in the optional concentration step d) by removing the stream 11, the latter being able to be recycled to step b). The organic raffinate 8 obtained on conclusion of step c), or alternatively the concentrated organic raffinate 10 obtained on conclusion of step d) if this is implemented, is treated in a hydrodistillation step e) in order to remove the residual organic solvent 13 and obtain the aqueous 5-HMF solution 12.

In this embodiment, the liquid-solid separation step f) is carried out during the liquid-liquid extraction, on an intermediate liquid stream withdrawn in the liquid-liquid extraction step b): the intermediate aqueous raffinate 19 produced in step b) is treated in the liquid-solid separation step f). This liquid-solid separation makes it possible to extract the solid, i.e. the precipitated humins, precipitated in the upstream phases, in the form of a stream of solid particles 18, and to produce a clarified intermediate aqueous raffinate 20 which is reincorporated into the extraction step b).

The aqueous raffinate 5 resulting from the liquid-liquid extraction step b) is sent to step g) of treating water-polar aprotic synthesis solvent mixtures. Step g) produces an aqueous effluent 15, an enriched polar aprotic synthesis solvent stream 16 and a heavy fraction stream 17 containing humins not removed by filtration and unconverted sugars (humins and sugars being in a form which is liquid albeit very viscous at the processing temperature of the mixture).

FIG. 2 illustrates another particular embodiment of the process according to the invention, which is identical to that represented in FIG. 1 except that the liquid-solid separation step f) is carried out downstream of the liquid-liquid extraction step b), on the aqueous raffinate obtained on conclusion of step b). In the embodiment represented, the precipitated humins forming solid particles are present in the aqueous raffinate obtained on conclusion of step b), which is sent to the liquid-solid separation step f) to separate said solid particles from the rest of the aqueous raffinate and produce a stream of solid particles 18 and said aqueous raffinate depleted in particles 5′. The latter is sent to step g) for the treatment of water-polar aprotic synthesis solvent mixtures, which functions in the same way as described for FIG. 1.

FIG. 3 illustrates another particular embodiment of the process according to the invention which is identical to that represented in FIG. 1, except that it includes the following specific features:
a liquid-solid separation step is carried out on the mixture 3 formed in step a) to separate precipitated humins (solid particles) in step a) from the rest of said mixture, producing a stream of solid 2;
the process comprises step d) of concentrating the organic raffinate 8 produced in the backwashing step c) to produce a concentrated organic raffinate 10 and a stream of extraction solvent 11, said stream of extraction solvent 11 advantageously being recycled to step b) and introduced with the extraction solvent 4;
the aqueous stream 21 used in mixing step a) comprises recycled water resulting from the process, in particular the aqueous stream 21 consists of the backwashing extract 9 and a fraction of the aqueous effluent 15 produced in step g) of treating water-polar aprotic synthesis solvent mixtures;
the aqueous solvent 7 used in the backwashing step c) consists of a fraction of the aqueous effluent 15 produced in step g) of treating the water-polar aprotic synthesis solvent mixtures and of the makeup water 22 (i.e. external to the process, in other words, neither produced nor resulting from a recycle in the process). Alternatively, the aqueous solvent 7 used in the backwashing step c) consists of a fraction of the aqueous effluent 15 produced in step g), without makeup water. In this case, the makeup water may be sent to step a) to form the aqueous stream 21 which comprises a fraction of the aqueous effluent 15 produced in step g). The supply of makeup water in the process may be necessary despite the recycles of water carried out;
the aqueous stream 14 used in the hydrodistillation step e) consists of a fraction of the aqueous stream 15 produced in step g) of treating water-polar aprotic synthesis solvent mixtures;
the extraction solvent 4 used in the extraction step b) is composed of the solvent streams produced in steps d) and e). In other words, the organic solvent streams 11 and 13 produced respectively in steps d) and e) are recycled to step b), and in particular form part of the composition of the extraction solvent 4.

This embodiment therefore comprises a recycle of the aqueous effluent 15 resulting from step g) of treating the water-polar aprotic synthesis solvent mixtures to steps a), c) and e), and a recycle of the organic solvent streams 11 and 13 to the liquid-liquid extraction step b), which firstly enables an optimal management of the water within the process with integrating its treatment within the process and avoiding excessive makeups of water, and secondly minimizes the consumption of extraction solvent, the combination of these ultimately having a favorable impact on the operating costs and the environmental impact of the process.

### LIST OF NUMERICAL REFERENCES USED IN THE FIGURES

1: feedstock
2: solid stream
3: aqueous mixture
4: extraction solvent
5: aqueous raffinate
6: organic extract
7: aqueous solvent
8: organic raffinate
9: intermediate aqueous back-extract
10: concentrated organic raffinate
11: first stream comprising organic solvent
12: aqueous 5-HMF solution
13: second stream comprising organic solvent
14: aqueous liquid
15: aqueous effluent
16: stream rich in polar aprotic synthesis solvent (or “residue”-rich . . . )
17: stream of impurities (or heavy fraction stream)
18: stream of solid particles
19: intermediate liquid stream
20: intermediate liquid stream depleted in particles
21: aqueous stream
22: makeup of water

### EXAMPLE

### Preparation of an Aqueous 5-HMF Solution 12 According to the Invention

The example below aims to show some of the advantages of the process according to the invention, performed according to the embodiment represented in FIG. 1.

An acid catalyst, methanesulfonic acid, is mixed with DMSO used as polar aprotic synthesis solvent, such that the molar ratio with the sugar feedstock (catalyst/sugar feedstock) is 1 mol %, and they are brought to a temperature of 120° C. Fructose is introduced in the form of an aqueous solution, at 70% by weight of sugar (syrup), in a DMSO/fructose mass ratio of 2.3. The pressure is maintained at 0.035 MPa. Under these pressure and temperature conditions, the reaction medium is above the bubble point of the mixture, so the vapor phase can be withdrawn from the reactor, and condensed to form the condensates. The sugar dehydration step is implemented batchwise, with addition of feedstock progressively over 2 h. The reaction medium is maintained at the temperature and pressure indicated above for a further 2 h after the end of the addition.

The liquid effluent resulting from the dehydration step contains 74% by weight of DMSO, 21% by weight of 5-HMF and 3% by weight of water, i.e. a molar yield of 5-HMF relative to the fructose employed of 81%. Polymeric compounds (called humins) which are soluble in the reaction medium were formed in an amount of 5% by weight. During this dehydration step, a water-DMSO mixture is recovered in the vapor phase. Said water-DMSO mixture has a composition of 32% by weight of DMSO and 68% water. This water-DMSO mixture is distilled under vacuum to produce water containing only traces of DMSO.

The liquid effluent resulting from the dehydration step corresponding to the feedstock 1 is employed in a step a) of bringing into contact with a water-containing stream, at ambient temperature, so as to obtain a mixture which contains a DMSO/water mass ratio equal to 1.

The mixture from step a) is subjected to a liquid-solid separation step, on a Büchner filter equipped with a polypropylene cloth filter with a pore size of 10 μm. This liquid-solid separation step is carried out at ambient temperature. During the liquid-solid separation step, 7.5 g of a “humins” solid residue/kg of filtered mixture are recovered, along with a homogeneous liquid phase corresponding to the aqueous mixture 3. The aqueous mixture 3 is composed of 43% by weight of DMSO, 12% by weight of 5-HMF and 43% by weight of water, and comprises impurities (about 2% by weight of humins). The aqueous mixture 3 resulting from step a) is subjected to a countercurrent liquid-liquid extraction step b) in a stirred glass column (Kühni or ECR type) comprising 8 sections 225 mm high with an inside diameter of 32 mm, and also a lower decanter and an upper decanter. The useful height is about 1.8 m and the total column height is 2.60 m. The total volume is about 3 liters. The organic extraction solvent is methyl isobutyl ketone (MIBK). Said aqueous mixture 3 is introduced into the upper part of the device and dispersed in the ascending organic phase. The column inlet flow rates are set at 2.2 kg/h for the DMSO-water phase and 4.1 kg/h for the MIBK. The proportion (weight/weight) of MIBK solvent is 1.9 relative to the aqueous mixture 3 resulting from step a). In this step b), the temperature is 20° C. and the stirring speed is 300 rpm.

The aqueous raffinate resulting from the liquid-liquid extraction column enters step f) which here is a filtration on a Buchner funnel equipped with a polypropylene cloth filter with a pore size of 1 μm. This liquid-solid separation step f) is carried out at ambient temperature. During the liquid-solid separation step f), 7.0 g of a “humins” solid residue/kg of filtered mixture are recovered, along with a homogeneous liquid phase corresponding to the aqueous raffinate depleted in solid particles, also referred to as clarified aqueous raffinate 20.

In this particular embodiment, the clarified aqueous raffinate 20 forms the aqueous raffinate 5 produced on conclusion of step b).

Recovered on conclusion of step b) are a 5-HMF-depleted aqueous raffinate 5, containing about 48% by weight of water, 48.5% by weight of DMSO, 0.4% by weight of 5-HMF, 1.8% by weight of MIBK, and humin impurities, and an organic extract 6 enriched in furan compounds containing 2.8% by weight of DMSO, 5.9% by weight of 5-HMF (i.e. a 5-HMF/DMSO weight ratio of about 68/32), and 91.3% by weight of MIBK. The extraction yield is 97% for 5-HMF and 13% for DMSO.

The aqueous raffinate 5 is treated in step g) for treating the water-DMSO mixture. The evaporation of the water and the DMSO is carried out sequentially in a batch distillation system with a first stage operated at a pressure of 0.008 MPa and 80° C. which makes it possible to recover the MIBK present in the aqueous raffinate 5 and almost all the water as well as a large part of the DMSO, until a DMSO content in the residue of 50% by weight is reached, i.e. an evaporation rate of about 93%. A second stage performed at 0.0005 MPa is stopped when the temperature of the medium reaches 90° C. At this stage, the heavy product contains only 20% by weight of DMSO and about 95% of the DMSO present in the aqueous raffinate 5 is recovered.

The organic extract 6 resulting from the liquid-liquid extraction step b) is subjected to a backwashing step c) in the same extraction device (ECR or Kühni type stirred column). Said organic extract is dispersed in the pure water phase at 21.5° C. The column inlet flow rates are set at 5 kg/h for the organic extract and 1.5 kg/h for the aqueous phase. The proportion (weight/weight) of water introduced as aqueous backwashing solvent relative to the organic extract is 0.3.

Recovered on conclusion of the backwashing step c) are a DMSO-enriched intermediate aqueous back-extract 9, containing 86% by weight of water, 7% by weight of DMSO, 5% by weight of 5-HMF and 2% by weight of MIBK, and an organic raffinate 8, containing 4.3% by weight of 5-HMF, 0.092% by weight of DMSO (i.e. 2.1% by weight of DMSO relative to the weight of 5-HMF) and 88% by weight of MIBK, giving a backwashing yield of 27% by weight for 5-HMF and 95% by weight for DMSO.

The organic raffinate 8 produced is sent to the concentration step d). The solvent is vaporized under vacuum. The temperature of the liquid is set at 60° C., and the vacuum pressure is set at 0.02 MPa.

Step d) is implemented with a vaporization mass ratio of 95%, corresponding to the mass of vaporized organic solvent relative to the employed mass of organic raffinate resulting from step c). The concentrated organic raffinate obtained on conclusion of step d) has a mass content of 84% by weight of 5-HMF, 2% by weight of DMSO and 9% by weight of MIBK. The 5-HMF content of the concentrated organic raffinate (84% by weight) is in accordance with the expected value (at least 40% by weight and at most 95% by weight), as is its residual solvent content of 11% by weight (sum of 9% MIBK +2% DMSO), which is in accordance with the expected value (at least 5% by weight and at most 60% by weight). The concentrated organic raffinate obtained on conclusion of step d) also comprises humin impurities (5% by weight). The recovered distillate contains essentially MIBK and water, removed in the form of an azeotrope with the MIBK, which separates into two immiscible phases on condensation.

The concentrated organic raffinate resulting from step d) is brought into contact with pure water, with a water/concentrated extract mass proportion of 0.95, and then sent into a hydrodistillation step e) implemented by distillation. The hydrodistillation step e) is implemented at a column bottom temperature of 35° C., and under a vacuum of 0.01 MPa, so as to facilitate the removal of residual MIBK organic solvent, in the form of a water/MIBK azeotrope without degradation of the 5-HMF. The aqueous 5-HMF solution obtained on conclusion of step e) has a composition of 45% by weight of 5-HMF, 53.3% by weight of water, 1% by weight of DMSO (i.e. 2.2% by weight of DMSO relative to the weight of 5-HMF) and 0.7% by weight of MIBK.

1. A process for producing an aqueous solution of hydroxymethylfurfural (5-HMF), said process comprising the following steps:
a step a) of bringing a feedstock (1) comprising 5-HMF and a polar aprotic synthesis solvent into contact with an aqueous stream (21) so as to obtain at least one aqueous mixture (3);
a step b) of liquid-liquid extraction of the aqueous mixture (3) obtained on conclusion of step a) in the presence of an extraction solvent (4), so as to produce an aqueous raffinate (5) comprising said polar aprotic synthesis solvent, an organic extract (6), a solid particles fraction, and optionally an intermediate liquid stream (19), said aqueous raffinate (5) and/or said intermediate liquid stream (19) comprising said solid particles fraction; then
a step c) of backwashing the organic extract (6) with an aqueous solvent (7), so as to produce an intermediate aqueous back-extract (9) and an organic raffinate (8) comprising 5-HMF and an organic solvent;
an optional step d) of concentrating said organic raffinate (8) resulting from step c) by removing at least a portion of the organic solvent, producing a concentrated organic raffinate (10) comprising 5-HMF and residual organic solvent, and producing a first stream (11) comprising organic solvent;
a hydrodistillation step e) implemented by distilling said organic raffinate (8) resulting from step c) or said concentrated organic raffinate (10) resulting from step d) in the presence of water, to produce an aqueous solution of 5-HMF (12) and a second stream (13) comprising organic solvent;
a step f) of liquid-solid separation of said solid fraction within said aqueous raffinate (5) resulting from step b) and/or said intermediate liquid stream (19) resulting from step b), producing a stream of solid particles (18) and an aqueous raffinate depleted in solid particles (5′) and/or an intermediate liquid stream depleted in particles (20) which is sent to step b); and
a step g) of treating at least one water-polar aprotic synthesis solvent mixture produced in said process, said mixture consisting of said aqueous raffinate depleted in particles (5′) and resulting from step f) or of said aqueous raffinate (5) not comprising the solid particles fraction and resulting from step b), to produce at least one aqueous effluent (15) that can be recycled into said process.

2. The process as claimed in claim 1, wherein step b) produces the intermediate liquid stream (19) comprising said solid particles fraction, and said intermediate liquid stream (19) is sent to step f) in order to separate said solid particles fraction from said intermediate liquid stream (19), forming said intermediate liquid stream depleted in particles (20) which is sent to step b), and at least said water-polar aprotic synthesis solvent mixture consisting of said aqueous raffinate (5) not comprising the solid particles fraction and resulting from step b) is sent to step g).

3. The process as claimed in claim 2, wherein said intermediate liquid stream (19) sent to step f) is an intermediate aqueous raffinate comprising said solid particles fraction and produced by separation between said extraction solvent and a water-polar aprotic synthesis solvent mixture in liquid-liquid extraction step b).

4. The process as claimed in claim 2, wherein said intermediate liquid stream (19) sent to step f) is a triphase mixture comprising a first liquid phase comprising 5-HMF and extraction solvent, a second liquid phase comprising water and polar aprotic synthesis solvent, and a solid phase comprising said solid particles fraction.

5. The process as claimed in claim 1, wherein said aqueous raffinate (5) produced in step b) comprises said solid particles fraction, and said aqueous raffinate (5) is sent to step f) in order to separate said solid particles fraction from said aqueous raffinate (5), forming said aqueous raffinate depleted in solid particles (5′) which is sent to step g) as water-polar aprotic synthesis solvent mixture.

6. The process as claimed in claim 1, wherein step f) is carried out at a temperature of between 0 and 60° C.

7. The process as claimed in claim 1, comprising step d) of concentrating the organic raffinate (8) resulting from step c) comprising vaporization of the organic solvent at atmospheric pressure or under vacuum, and a liquid temperature maintained at less than or equal to 130° C., said concentrated organic raffinate (10) comprising 5-HMF at a content of greater than or equal to 40% by weight and residual organic solvent at a content of less than or equal to 60% by weight.

8. The process as claimed in claim 1, wherein step e) is carried out at atmospheric pressure or under vacuum, and preferably under vacuum at a pressure of between 0.005 MPa and 0.08 MPa.

9. The process as claimed in claim 1, wherein step e) is implemented in a distillation column.

10. The process as claimed in claim 1, wherein the extraction solvent (4) is chosen from dichloromethane, diethyl ether, diisopropyl ether, methyl ethyl ketone, methyl isopropyl ketone, methyl isobutyl ketone, thiophene, anisole, and toluene.

11. The process as claimed in claim 1, wherein the weight ratio (weight/weight) of aqueous solvent (7) relative to the organic extract (6) in backwashing step c) is between 0.04 and 5.

12. The process as claimed in claim 1, comprising a step of dehydrating sugars to 5-HMF upstream of step a).

13. The process as claimed in claim 1, wherein said aqueous effluent produced in step g) is used in full or in part in step a) and/or in step c) and/or in step e).

14. The process as claimed in claim 1, wherein in step a) said aqueous stream (21) comprises all or a fraction of said intermediate aqueous back-extract (9) resulting from step c).

15. The process as claimed in claim 1, wherein said polar aprotic synthesis solvent is chosen from pyridine, butan-2-one, acetone, acetic anhydride, N,N,N′,N′-tetramethylurea, benzonitrile, acetonitrile, methyl ethyl ketone, propionitrile, hexamethylphosphoramide, nitrobenzene, nitromethane, N, N-dimethylformamide, N, N-dimethylacetamide, sulfolane, N-methylpyrrolidone, dimethyl sulfoxide, propylene carbonate, and γ-valerolactone, taken alone or as a mixture, and is preferably dimethyl sulfoxide.

16. The process as claimed in claim 1, wherein step f) is carried out at a temperature of between 0 and 60° C., and comprises filtration carried out by a filter press.

17. The process as claimed in claim 1, comprising step d) of concentrating the organic raffinate (8) resulting from step c) comprising vaporization of the organic solvent at a pressure of between 0.01 MPa and 0.1 MPa, and a liquid temperature maintained at less than or equal to 130° C., said concentrated organic raffinate (10) comprising 5-HMF at a content of greater than or equal to 40% by weight and residual organic solvent at a content of less than or equal to 60% by weight.

18. The process as claimed in claim 1, wherein step e) is carried out at a pressure of between 0.001 MPa and 0.1 MPa.

19. The process as claimed in claim 1, wherein step e) is implemented in a distillation column at a column bottom temperature of less than or equal to 140° C.

20. The process as claimed in claim 1, comprising a step of dehydrating sugars to 5-HMF upstream of step a) by bringing a sugar feedstock comprising one or more sugars into contact with said polar aprotic synthesis solvent and a dehydration acid catalyst at a temperature of between 30° C. and 200° C. and at a pressure of between 0.001 MPa and 10 MPa.
